(19) United States
(12) Patent Application Publication
(10) Pub. No.: US 2010/0249585 A1
(43) Pub. Date: Sep. 30, 2010

(54) Power Injector Syringe Mounting System

(76) Inventor: Geoffrey S. Strobl, Williamsburg, OH (US)

Correspondence Address: Mallinckrodt Inc., 675 McDonnell Boulevard, HAZELWOOD, MO, 63042, US

(21) Appl. No.: 12/739,412

(22) PCT Filed: Dec. 2, 2008
(86) PCT No.: PCT/US08/85229

§ 371 Date: Apr. 23, 2010

Related U.S. Application Data
(60) Provisional application No. 61/012,091, filed on Dec. 7, 2007.

(51) Int. Cl. A61B 6/00

(52) U.S. Cl. 600/432

(57) ABSTRACT

The disclosed embodiments provide for a syringe mount for a medical fluid power injector. The syringe mount is designed to hold a syringe so that a drive of the medical fluid power injector can move a plunger of the syringe to expel medical fluid from the syringe. The syringe mount comprises a first syringe clamp comprising a first arcuate surface, and a second syringe clamp comprising a second arcuate surface, wherein the first and second syringe clamps are in opposing relation to one another such that the first arcuate surface faces the second arcuate surface, and wherein the first and second syringe clamps are adapted to translate in opposite linear directions toward and away from one another to engage and disengage a syringe located therebetween.

### RELATED APPLICATIONS

This application claims priority to U.S. Provisional Patent Application No. 61/012,091 filed on 7 Dec. 2007 and entitled “Power Injector Syringe Mounting System”.

### FIELD OF THE INVENTION

The invention relates generally to powered injectors for injecting medical fluids and, more specifically, to systems and methods for mounting syringes onto powered injectors.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Generally, a power injector is used to inject medical fluids, such as a radiopharmaceutical or contrast media, into a patient. For example, the medical fluid may be disposed within a syringe, which in turn may be mounted onto the power injector. When injecting the medical fluid into the patient, a ram disposed behind the mounted syringe may push the syringe's plunger, thereby forcing the fluid toward the tip of the syringe to inject the fluid into the patient. Unfortunately, existing power injectors may suffer from design shortcomings which may affect the manner by which syringes may be mounted onto and/or may be retained within a power injector. Such design inadequacies may lower overall quality and/or efficiency of the injection process. In addition, design shortcomings associated with power injectors may complicate the manner by which a user, such as a healthcare provider, administers an injection procedure.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A first aspect of the present invention is directed to front-loading, contrast media injector (e.g., for use in a medical imaging procedure such as a CT, MR, PET, SPECT, ultrasound, optical, or other medical imaging procedure). This injector includes a drive ram and an injector housing, which has an opening defined therein through which the drive ram is designed to pass. In other words, at least a portion of the drive ram is movable into and out of the injector housing (e.g., along a longitudinal reference axis of the drive ram) through the opening in the injector housing. The injector also includes a syringe mount that is coupled to the housing and that at least assists in holding a syringe so that the injector can be utilized to expel an appropriate medical fluid (e.g., contrast media, saline, or a combination thereof) from the syringe. This syringe mount includes a first syringe clamp that has a first arcuate surface. In addition, the syringe mount also includes a second syringe clamp that has a second arcuate surface. These first and second syringe clamps are in opposing relation to one another such that the first arcuate surface faces the second arcuate surface. Moreover, the first and second syringe clamps translate in opposite linear directions toward and away from one another to engage and disengage a syringe located therebetween.

Various refinements exist of the features noted in relation to the first aspect of the present invention. Further features may also be incorporated in the first aspect of the present invention as well. These refinements and additional features may exist individually or in any combination. The following discussion pertains to this first aspect, up to the start of the discussion of a second aspect of the present invention.

The syringe clamps may exhibit any appropriate design as long as they at least assist in allowing a syringe to be mounted to the injector. For instance, the first and second syringe clamps may be designed such that an aperture is defined therebetween to align a plunger of a syringe with the drive ram of the injector. As such, the first and second syringe clamps may be configured to open and close about a circumferential portion of a syringe. In some embodiments, the first and second syringe clamps may contact one another when the same are closed about a circumferential portion of a syringe. Each of these first and second syringe clamps may include or be characterized by some as a C-clamp of sorts.

The syringe mount may be designed in any of a number of appropriate fashions to enable translation of the first and second syringe clamps toward and away from each other. For instance, syringe mount may be designed such that the first and second syringe clamps move along rails of the syringe mount between a syringe-engaging position and a syringe-disengaging position. In some embodiments, the syringe mount may include a lever configured to actuate the first and second syringe clamps between the syringe-engaging position and the syringe-disengaging position.

Some embodiments of the syringe mount may be design to enable the first and second syringe clamps to substantially provide full support for a syringe that is mounted to the injector. Other embodiments of the syringe mount may include one or more additional components to at least assist in supporting a mounted syringe. For instance, some embodiments of the syringe mount may include an elongate syringe support extending out away from the injector housing.

The syringe mount may be coupled to the injector housing in any appropriate manner. Incidentally, “coupled” or the like herein refers to a condition of one thing being at least temporarily connected (either directly or indirectly) with another thing. As an example of an appropriate coupling of the syringe mount and the injector, the syringe mount may be a component of a removable face plate of the injector. As another example, the syringe mount may be substantially integral with the injector housing. As yet another example, the syringe mount may be a component of an adapter for at least temporarily making an original syringe mount of the injector compatible to accommodate a syringe that the original syringe mount was not originally designed to accommodate.

A second aspect of the invention is directed to a method of using (e.g., mounting a syringe to) a powered contrast media injector. In this method, a rearward portion of a syringe is disposed between first and second syringe clamps of the injector, each of which includes an arcuate surface. Further, the first and second syringe clamps are translated (e.g., laterally) toward one another at least until each of the arcuate surfaces contacts the rearward portion of the syringe.

Various refinements exist of the features noted in relation to the second aspect of the present invention. Further features may also be incorporated in the second aspect of the present invention as well. These refinements and additional features may exist individually or in any combination. The following discussion pertains to this second aspect, up to the start of the discussion of a third aspect of the present invention.

The first and second syringe clamps may be translated until they are in direct contact with one another. In some embodiments, the relative positions of the first and second syringe clamps may be selectively locked (e.g., as a user desires). For instance, a lock may be actuated to substantially immobilize the first and second syringe clamps after the first and second syringe clamps are translated toward one another.

In some embodiments, a user may cause the injector (e.g., through programming and/or manual activation) to move a plunger of the syringe while the rearward end of the syringe is disposed between the first and second syringe clamps. For instance, contrast media and/or saline may be expelled from the syringe due to this movement of the plunger.

Yet a third aspect of the invention is directed to a method of using (e.g., removing a mounted syringe from) a powered contrast media injector. In this method, first and second syringe clamps of the injector are disposed about and in contact with a rearward portion of a syringe. The first and second syringe clamps are translated (e.g., in opposite lateral directions) away from the syringe that is located therebetween. Thereafter, the syringe is moved out from between the first and second syringe clamps.

Various refinements exist of the features noted in relation to the third aspect of the present invention. Further features may also be incorporated in the third aspect of the present invention as well. These refinements and additional features may exist individually or in any combination. The following portion of the summary pertains to this third aspect of the present invention.

The first and second members may be in direct contact with one another prior to being translated away from the syringe. In some embodiments, the injector may include a clamp lock. In such embodiments, the clamp lock may be unlocked (e.g., by a user) to allow for translation of the first and second syringe clamps. This unlocking of the clamp lock may occur at any appropriate time. For instance, in some embodiments, the clamp lock may be unlocked prior to the first and second syringe clamps being translated away from the syringe.

Again, the brief summary presented above is intended only to familiarize the reader with certain aspects and contexts of the present invention without limitation to the claimed subject matter.

### BRIEF DESCRIPTION OF THE FIGURES

Various features, aspects, and advantages of the present invention will become better understood when the following detailed description is read with reference to the accompanying figures in which like characters represent like parts throughout the figures, wherein:

FIG. 1 is a block diagram of an imaging system;

FIG. 2 is a perspective view of a power injector;

FIG. 3 is a top view of a syringe mounting system;

FIG. 4 is a perspective view of the syringe mounting system of FIG. 3 having a syringe mounted thereto;

FIG. 5 is another perspective view of the syringe mounting of FIG. 3;

FIG. 6 is a top view of a dual head syringe mounting system; and

FIG. 7 is a flow chart of a syringe loading process.

### DETAILED DESCRIPTION OF SPECIFIC EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top”, “bottom”, “above”, “below” and variations of these terms is made for convenience, but does not require any particular orientation of the components.

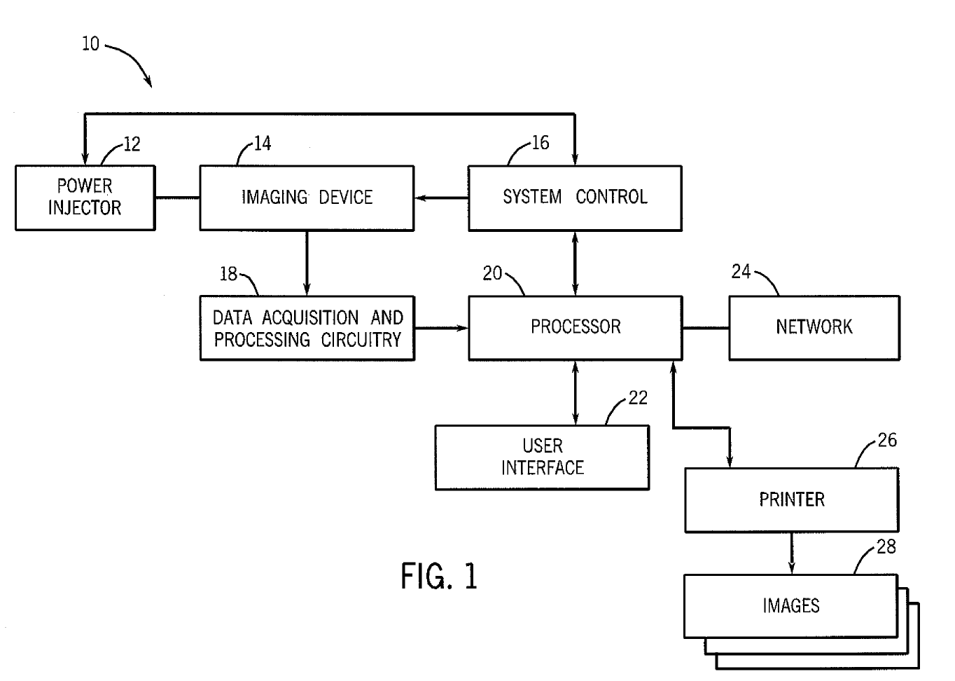

Referring to FIG. 1, an imaging system 10 may include a power injector 12 adapted to inject medical fluids from a syringe and/or a plurality of syringes loaded onto the power injector 12. The medical fluid injected from syringes using the power injector 12 may include any appropriate medical fluid (e.g., contrast media, saline solution, or a combination thereof). The imaging system 10 further includes an imaging device 14, a system control 16, data acquisition and processing circuitry 18, a processor 20, a user interface 22, and a network 24. Specifically, the imaging device 14 is configured to obtain signals representative of an image of a subject after and/or during administration of the medical fluid (e.g., contrast media) to the subject via the power injector 12. The imaging system 10 may include a positron emission tomography (PET) system, a single photon emission computer tomography (SPECT) system, a nuclear medicine gamma ray camera, a magnetic resonance imaging (MRI) system, a computerized tomography (CT) system, an X-ray camera, an optical imaging system, an ultrasound imaging system, or another suitable imaging modality. Image data indicative of regions of interest in a subject may be created by the imaging device 14 either in a conventional support, such as photographic film, a digital medium, or other appropriate fashion.

The system control 16 may include a wide range of circuits, such as imaging (e.g., radiation) source control circuits, timing circuits, circuits for coordinating data acquisition in conjunction with patient or table movements, circuits for controlling the position of imaging (e.g., radiation) detectors, and so forth. The imaging device 14, following acquisition of the image data or signals, may process the signals, such as for conversion to digital values, and forward the image data to the data acquisition circuitry 18. In the case of analog media, such as photographic film, the data acquisition system may generally include supports for the film, as well as equipment for developing the film and producing hard copies that may be subsequently digitized. For digital systems, the data acquisition circuitry 18 may perform a wide range of initial processing functions, such as adjustment of digital dynamic ranges, smoothing or sharpening of data, as well as compiling of data streams and files, where desired. The data is then transferred to a processor 20 where additional processing and analysis is performed. For conventional media such as photographic film, the processor 20 may apply textual information to films, as well as attach certain notes or patient-identifying information. In a digital imaging system, the data processing circuitry may perform substantial analyses of data, ordering of data, sharpening, smoothing, feature recognition, and so forth.

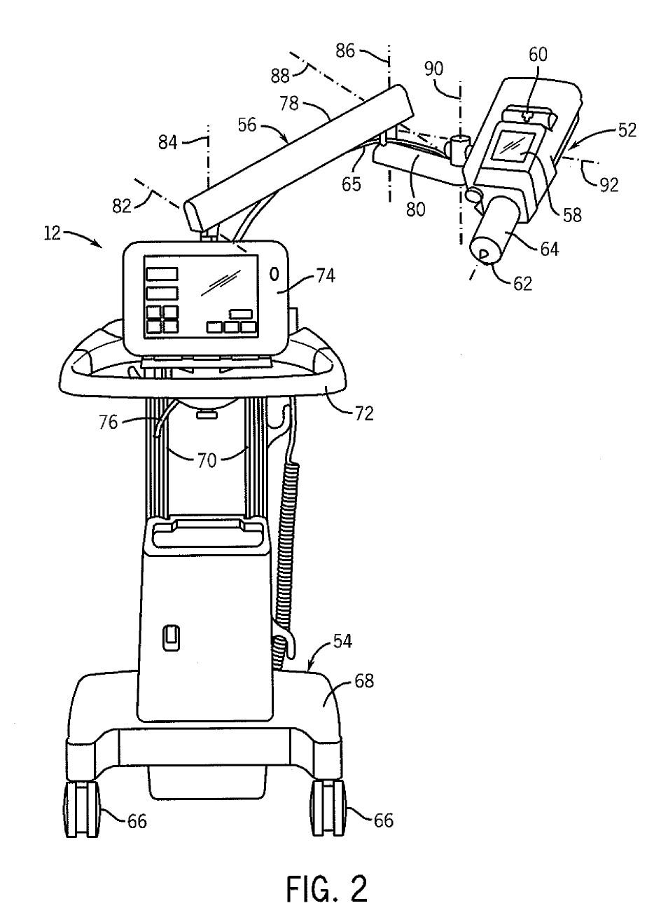

Ultimately, the image data may be forwarded to an operator/user interface 22 for viewing and analysis. While operations may be performed on the image data prior to viewing, the operator interface 22 is at some point useful for viewing reconstructed images based upon the image data collected. In the case of photographic film, images may be posted on light boxes or similar displays to permit radiologists and attending physicians to more easily read and annotate image sequences. The image data can also be transferred to remote locations, such as via a network 24. In addition, the operator interface 22 may enable control of the imaging system (e.g., by interfacing with the system control 16). Furthermore, the imaging system 10 may include a printer 26 to output a hard copy of images 28. While FIG. 1 shows an example of one imaging system 10, it should be noted that principles of the invention apply to any imaging system utilizing an injector that includes or should include a syringe mount. Further, while FIG. 2 shows an exemplary injector 12, it should be noted that principles of the invention apply to any medical fluid injector that includes or should include a syringe mount.

FIG. 2 is a perspective view of the exemplary power injector 12 diagrammatically represented in FIG. 1. As illustrated by FIG. 2, the power injector 12 may include a powerhead 52, a stand assembly 54, and a support arm 56. Powerhead 52 may include a syringe mounting system adapted to aptly capture a syringe containing a medical fluid. For example, such a mounting system may provide optimal fluid injection parameters, such as pressure provided by the powerhead's drive ram to the syringe's plunger, for injecting fluid at a desired rate. Syringe mounting systems included with powerhead 52 may be adapted to simplify tasks associated with loading syringes onto the powerhead 52, which may further enable the clinician to attend to additional tasks associated with other aspects of the injection procedure. The powerhead 52 may include a display 58, a fluid control bar 60, and an air detector 62. The fluid control bar 60 may facilitate manual manipulation of a plunger in a syringe 64 mounted to the powerhead 52. The air detector 62 may signal a controller, such as the controller 16 (FIG. 1) when air is detected in or leaving syringe 64.

The illustrated stand assembly 54 includes a set of four wheels 66, a chassis 68, vertical supports 70, a handle 72, and a display 74. The vertical supports 70 may adjustably elevate handle 72, display 74, and the support arm 56 above the chassis 68, and, in certain embodiments, it may have a recessed portion through which power cable 76 is routed. The display 74 may include a liquid crystal display, a cathode ray tube display, an organic light emitting diode display, a surface emission display, or other appropriate display. The support arm 56 of the injector 12 shown in FIG. 2 includes multi-axis articulating members 78, 80. The illustrated articulating member 78 has two degrees of freedom relative to the chassis 68 due to two perpendicular axes of rotation 82, 84. Similarly, the exemplary articulating member 80 has two degrees of freedom relative to the articulating member 78 by virtue of two perpendicular axes of rotation 86, 88. The power cable 76 is shown as being routed along the articulating members 78, 80 to the powerhead 52.

The powerhead 52 of FIG. 2 may couple to the articulating member 80 via a joint that provides two degrees of freedom relative to the articulating member 80. As a result, in the present embodiment, the powerhead 52 may rotate about the axes 90, 92. In total, the illustrated powerhead 52 has six degrees of freedom relative to the chassis 68. Other embodiments may include more or fewer degrees of freedom.

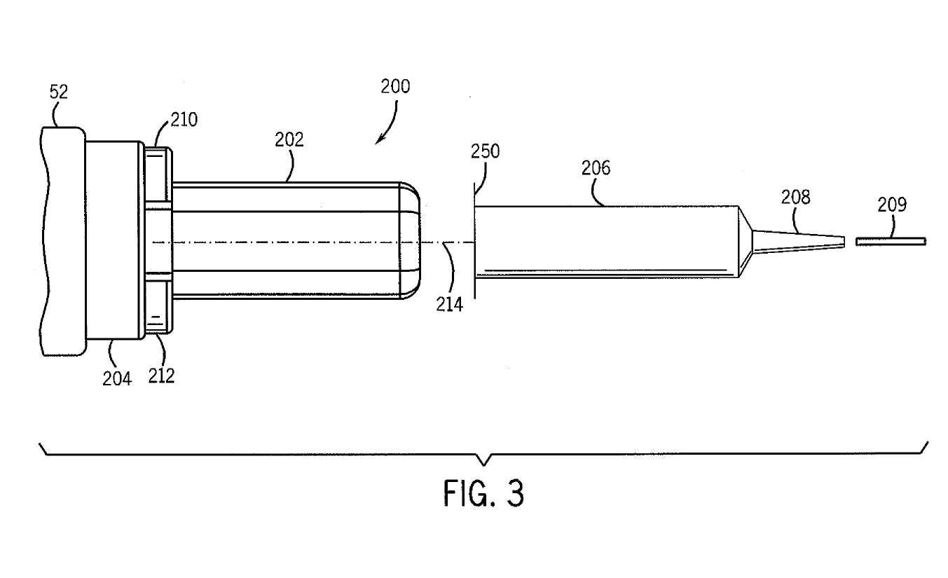

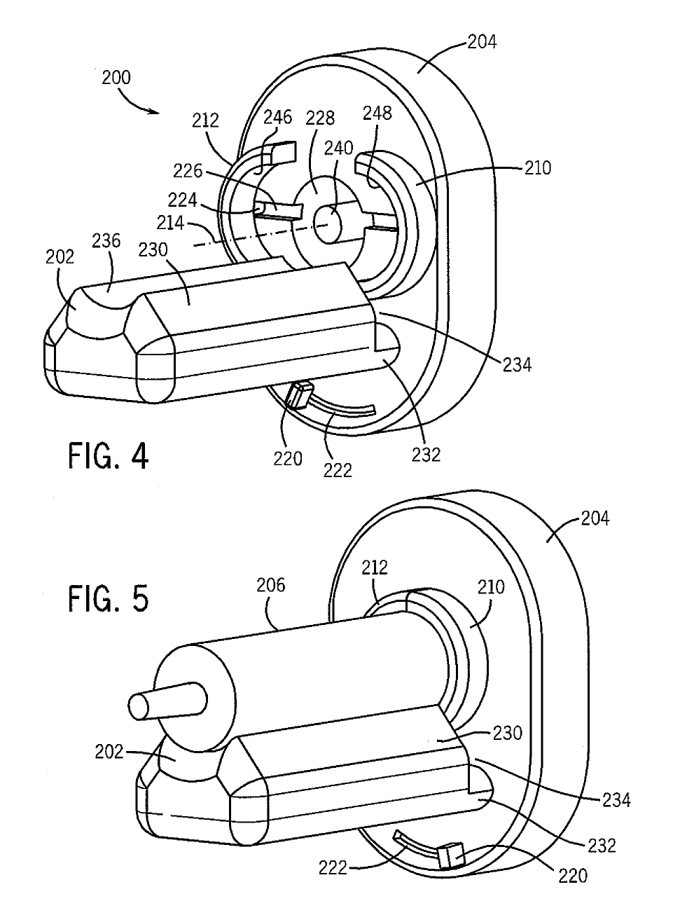

FIGS. 3-5 show a syringe mounting system 200 for use with a medical fluid power injector, such as power injector 12 (FIG. 1). The syringe mounting system 200 includes an elongate syringe support or cradle 202 and a base 204. The base 204 is coupled to both the cradle 202 and the powerhead 52, such that a syringe 206 can be mounted in close proximity and alignment with the powerhead 52. The syringe mounting system 200 also includes first and second clamps 210, 212 coupled to the base 202 at a fixed end of the elongate cradle 202. As discussed in detail below, the cradle 202 may support the syringe 206 (e.g., through direct contact of a barrel of the syringe 206 with the cradle 202) in a manner allowing axial movement of the syringe 206 along a longitudinal axis 214, while generally deterring at least some non-axial movement of the syringe 206. In turn, the clamps 210, 212 may secure the syringe 206 in the cradle 202, thereby hindering the syringe 206 from moving significantly axially along the longitudinal axis 214 and non-axially (e.g., vertically (e.g., perpendicular to the sheet of FIG. 3) and/or laterally) out of the cradle 202. Thus, the cradle 202 and clamps 210, 212 may cooperatively support and secure the syringe 206 to the base 204 and the powerhead 52, such that the syringe 206 is coaxial (e.g., axially aligned) with the powerhead 52 along the longitudinal axis 214. The axial alignment provided by the syringe mounting system 200 may promote a drive ram 240 (FIG. 4) of the powerhead 52 moving a plunger of the syringe 206 to expel medical fluid from the syringe 206 and optionally to draw medical fluid into the syringe (e.g., via an appropriate interfacing of the drive ram 240 with the plunger of the syringe).

As illustrated, the cradle 202 extends away from (e.g., perpendicular to) the base 204 and the powerhead 52. The cradle 202 may substantially extend or protrude out from the base 204 (e.g., in a generally perpendicular direction). As such, the cradle 202 may be described as cantilevered relative to the powerhead 52 and/or the base 204. In other words, the cradle 202 may be fixed to the powerhead 52 and/or the base 204 at a first end (e.g., fixed proximate end), while an opposite second end of the cradle 202 is free standing or not fixed (e.g., free peripheral end).

The cradle 202 may include an open top configured to enable insertion of the syringe 206 from above the cradle 202 to a position on top of the cradle 202. For example, an upper surface 236 of the cradle 202 may have an upper syringe receptacle defined therein that extends along a portion (e.g., up to a substantial entirety) of a length the syringe 206. As further discussed below, the upper surface 236 of the cradle 202 may be shaped to conform to the shape and contour of at least a portion of the syringe 206. For example, the cradle 202 may have a curved, recessed, upper surface 236 (e.g., a semi-cylindrical, recessed surface), which is accessible from above the cradle 202 and also from the free peripheral end. The curved recess may be adapted to receive and fit about a bottom portion of the syringe 206 along at least a portion of length thereof. For example, the cradle 202 may contact only the bottom portion of the syringe 206, while not extending over, covering, or otherwise contacting a top portion of the syringe 206. The cradle 202 may be adapted to provide proper support for the syringe 206, particularly, during syringe filling and injection procedures involving a medical fluid and the syringe 206. The support provided by the cradle 206 may assist in preventing the weight of syringe 206 and/or the weight of the medical fluid disposed therein from causing the syringe 206 to bend, brake or otherwise suffer any shape deformation that could hamper injection of medical fluid from the syringe 206. Similarly, the support provided by the cradle 202 for retaining the syringe 206 to base 204 may assist in enabling the drive ram 240 of the powerhead 52 to apply optimal injection pressure to the plunger of the syringe 206 as medical fluid is injected from the syringe 206 into a patient.

The syringe mounting system 200 may have an aperture that is defined between first and second syringe clamps 210, 212 thereof to assist in aligning the plunger of the syringe 206 with the drive ram 240 of the powerhead 52. The drive ram 240 may interface with (e.g., engage) and apply pressure to the plunger of the syringe 206 in any appropriate fashion, thereby moving the medical fluid outward through a tip 208 of the syringe and tube 209 leading to a patient. As a result, alignment may be important to promote a desired interface between the drive ram of the powerhead 52 and the plunger in the syringe 206. Accordingly, the user may place the syringe 206 on top of the cradle 202 so as to at least generally align the syringe 206 with the central axis 214 and the drive ram 240 of the powerhead 52. In other words, the syringe 206 and the drive ram 240 of the powerhead 52 may be coaxial with one another in alignment with the longitudinal axis 214. In some embodiments, the cradle 202 is sized to fit with a particularly sized syringe 206, thereby promoting precise alignment of that particular syringe 206 with the powerhead 52. For example, a semi-cylindrical groove in the cradle 202 may have the same or substantially similar arcuate contour as the cylindrical exterior of the syringe 206. In other embodiments, the cradle 202 may include a plurality of different sized grooves, each adapted to accommodate a differently sized syringe 206. For example, a plurality of semi-cylindrical grooves may be recessed one inside another along the top surface of the cradle 202. In other embodiments, the system 200 may include a plurality of replaceable cradles 202 having differently sized semi-cylindrical grooves, such that the cradle 202 can be quickly replaced to accommodate a particular syringe 206.

The syringe mounting system 200 also includes first and second syringe clamps 210, 212 that may be utilized to provide retention and/or alignment functions. The clamps 210, 212 may be characterized as a pair of retaining structures (e.g., C-clamps) that may be used in securing the syringe 206 to the syringe mounting system 200. The first syringe clamp 210 is shown as having a first arcuate surface 246; similarly, the second syringe clamp 212 is shown as having a second arcuate surface 248. The clamps 210, 212 are in opposing relation to one another such that the first arcuate surface 246 faces the second arcuate surface 248. The clamps 210, 212 are adapted to translate in opposite linear directions toward and away from one another to engage and disengage the syringe 206 located therebetween. The first and second arcuate surfaces 246, 248 may be designed such that they, in combination, at least substantially surround and conform to an outer diameter of the syringe 206 that is located therebetween.

The clamps 210, 212 are movable, such as by a lever as discussed further below, along an axis perpendicular to central axis 214 of the syringe mounting assembly 200. The clamps 210, 212 are adapted to translate (e.g., move linearly without rotation) toward and away from one another. Accordingly, attachment or detachment of the syringe 206 to or from the syringe mounting assembly 200 may be facilitated by moving the clamps 210, 212 in opposite directions along a line that is substantially perpendicular to the axis 214. As illustrated, prior to mounting the syringe 206 on the syringe mounting system 200, the clamps 210, 212 may be placed in an open condition (e.g., disposed apart from one another) such that their outer edges approximately align with the edges of the base 204. In this open condition, the first syringe clamp 210 is separated from the second syringe clamp 212 by a first distance. In contrast, the first syringe clamp 210 is separated from the second syringe clamp 212 by a second distance, which is less than the first distance, when in a closed position.

To retain the syringe 206 to the syringe mounting system 200, the clamps 210, 212 may be actuated to move linearly toward each other until the clamps 210, 212 contact the outer circumferential portion of the syringe 206. Thus, the clamps 210, 212 are configured to close about a circumferential portion of the syringe 206. In so doing, a portion of the syringe 206 becomes disposed or captured between the clamps 210, 212. Moreover, upon closure of the clamps 210, 212, a retaining flange 250 of the syringe is located behind the clamps 210, 212 (e.g., trapped between the base 204 and the clamps 210, 212) to hinder movement of the syringe 206 along the axis 214.

The syringe mounting system 200 may be adapted to hold the syringe 206 for relatively prolonged periods of time (e.g., as facilitated by the cradle 202 and the clamps 210, 212), as may be desired in certain injection procedures. Accordingly, the syringe mounting system 200 may be adapted to preserve the structural integrity of the syringe 206 while it is mounted on the syringe mounting system 200. For example, the cradle 202 and the clamps 210, 212 may be adapted to counter structural faults, bents or material fatigue which otherwise could manifest without the support provided by structures. This may reduce or limit fluid spillages stemming from any of the aforementioned syringe failures, consequently, reducing the amount of time devoted to cleaning and/or restoring the work environment where injection is performed. Further, the proper retention and alignment of syringes, as provided by the cradle 202 and the clamps 210, 212, may reduce syringe movement which could interfere with the desired results of an injection procedure.

As illustrated in FIGS. 4-5, the syringe mounting system 200 may include a lever 220 disposed beneath the cradle 202. The lever 220 is adapted to slide along a rail 222 (e.g., an arcuate slot), as lever 220 moves between an open position and a closed position. The clamps 210, 212 may be coupled to the base 204 via movable tabs 224 disposed within rails 226 of the base 204. The clamps 210, 212 are adapted to translate (e.g., move linearly without rotation) along rails 226 of the syringe mounting system 200. The illustrated rails 226 may be characterized as two opposing narrow structures defined in the front face of the base 204. The rails 226 are shown as extending from outer edges of the base 204 to outer edges of an aperture 228 defined centrally in the base 204. Other embodiments may exhibit other appropriate rail design for controlling/guiding movement of the clamps 210, 212. The movable tabs 224 are designed to move along the rails in a fashion enabling the clamps 210, 212 to translate toward and away from one another, so that the clamps 210, 212 may engage the syringe 206 when disposed on the cradle 202 to thereby mount the syringe 206 on the syringe mounting system 200. It should be born in mind that while the present embodiment employs a single pair of clamps 210, 212, other embodiments may employ multiple pairs of clamps.

Referring to FIG. 4, the cradle 202 may be characterized has having two portions, namely, an upper portion 230 and a lower portion 232. The lower portion 232 of the cradle 202 is coupled to the base 204, such that lower portion 232 extends from the base 204 to the outer edge of the cradle 202. The upper portion 230 of the cradle 202 extends approximately from the outer edges of the clamps 210, 212 to the outer edge of cradle 202. As such, the upper portion 230 of the cradle 202 is not coupled to the base 204, thereby forming a gap 234 between the cradle 202 and the base 204. This enables the clamps 210, 212 to freely move along the face of the base 204, while the cradle 202 is securely coupled to the base 204.

As mentioned above, the user may actuate the clamps 210, 212 to lock or unlock the syringe 206 to or from the syringe mounting system 200 via a locking mechanism (not shown) that may include the lever 220. For example, in one embodiment, the lever 220 may be coupled to a mechanism (e.g., a rotatable disk, spring-loaded mechanism, etc.) disposed within the base 204. This mechanism may interface (e.g., contact or couple) with the movable tabs 224 such that movement of the lever 220 between the open position and the closed position causes movement of the clamps 210, 212 between the corresponding open condition and closed condition.

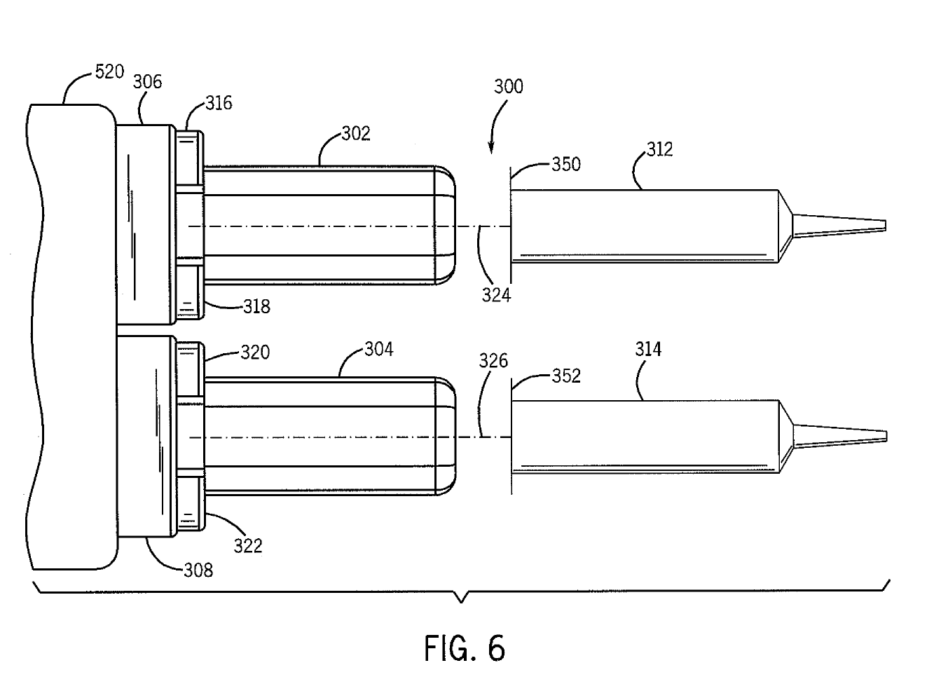

FIG. 6 is a top view of a syringe mounting system 300 of a dual-head, contrast media injector 520. Syringe mounting system 300 is a dual syringe mounting system, which may be adapted to receive two syringes for injecting medical fluids therefrom. Accordingly, the syringe mounting system 300 may include any of the features discussed with regard to the syringe mounting system 200 shown in FIGS. 3-5. For example, the syringe mounting system 300 includes cradles 302, 304, coupled to bases 306, 308, respectively.

It should be noted that while the present embodiment may illustrate cradles 302, 304 as having the same size, other embodiments may include syringe mounting systems having a plurality of cradles of various sizes. In some embodiments, for example, the cradle 302 may accommodate a syringe (e.g., syringe 312) of a first size, while the cradle 304 may accommodate a syringe (e.g., syringe 314) of a second size different from the first size.

Each of the bases 306, 308 are coupled to a pair of clamps. In particular, the base 306 is coupled with clamps 316, 318, and the base 308 is coupled with clamps 320, 322. Clamps 316, 318, 320, 322 are adapted to securely mount the syringes 312, 314, respectively, to the syringe mounting system 300. Each pair of the clamps 316, 318 and 320, 322 may be actuated, for example, by a lever adapted to move each pair of clamps between open and closed conditions like that described above with regard to FIGS. 3-5. In the illustrated embodiment, each pair of the clamps 316, 318 and 320, 322, may be independently actuated for retaining or releasing each of the syringes 312 and 314, respectively. In other embodiments, the locking mechanism(s) of the syringe mounting system 300 may be designed to such that clamps 316, 318, 320, 322 are required to translate simultaneously in tandem for securing or releasing the syringes 312, 314 relative to syringe mounting system 300.

The syringe mounting system 300 may be a component of a removable faceplate for the power injector 520. For instance, bases 306, 308 may each independently couple to the injector 520 (e.g., via latches, fasteners or other coupling mechanisms) to enable removal and/or replacement of the bases 306 and/or 308 with other similar bases adapted to accommodate syringes of different types and sizes. In other embodiments, the syringe mounting system 300 may be integral with the power injector 520. In yet other embodiments, the syringe mounting system 300 may be components of an adapter system for at least temporarily making an original syringe mounting system of a dual-head injector compatible to accommodate syringes that the original syringe mounting system was not originally designed to accommodate.

The illustrated syringe mounting system 300 may be desirable for use in various settings, such as those employing simultaneous and/or sequential injection of medical fluids. To accommodate such settings, the injector 520 may be configured to inject medical fluids from each of the syringes 312, 314 independently. For example, the injector 520 may include multiple rams, whereby each ram may be configured to apply a desired amount of pressure to the respective plungers of the syringes 312, 314.

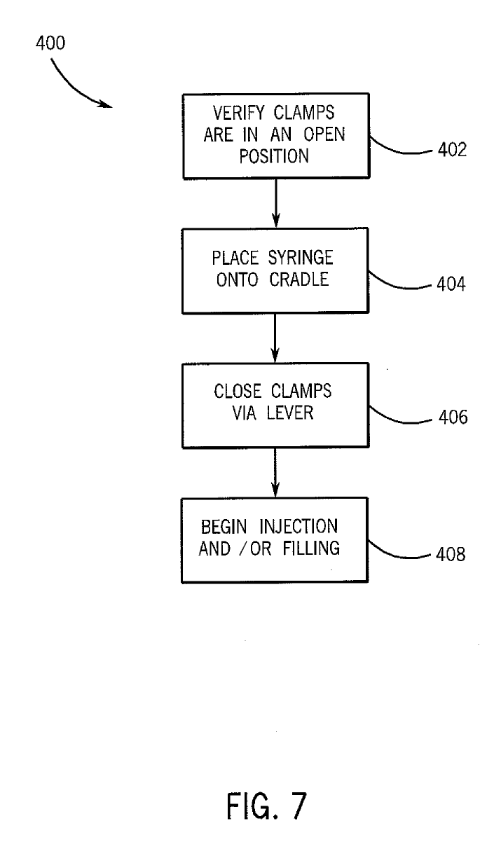

FIG. 7 shows an exemplary process for mounting and/or loading a syringe onto the syringe mounting system 200 discussed in relation to FIGS. 3-6. This process 400 may be employed by a user, such as healthcare provider, administering an injection procedure, whereby a patient is injected with medical fluid used for imaging desired anatomical structures and/or physiological processes of the patient. For example, the medical fluid may include a radiopharmaceutical, contrast media, saline solution, or a combination thereof. The process 400 begins at step 402, whereby the user may verify that the syringe mounting system 200 is in an open condition (e.g., clamps 210, 212 are appropriately spaced apart) in order to receive a syringe (see FIG. 4). Thereafter, the process 400 proceeds to step 404 in which the user may place a fluid filled syringe, such as the syringe 206 of FIG. 5, onto the cradle 202. For instance, the user may dispose a rearward portion of a syringe 206 between the first and second syringe clamps 210, 212 such that the flange 250 of the syringe 206 will be located behind the clamps 210, 212 upon the clamps being moved to the closed condition (see FIG. 5). Thereafter, step 406 follows whereby the syringe may be securely coupled to the syringe mounting system 200 by the pair of clamps 210, 212 being moved to the closed condition in which they are disposed about and contact a rearward portion of the syringe. To achieve this closed condition, the user may translate the first and second syringe clamps 210, 212 toward one another until each of the arcuate surfaces 246, 248 contacts the rearward portion of the syringe 206. As at least generally described above with regard to FIGS. 4-5, this step 406 may be initiated by actuating the lever 220. At this point, the syringe 206 is properly mounted on the syringe mounting system 200 so that an injection and/or filling procedure may commence, as provided by final step 408. In some embodiments, the above method may include the step of actuating a lock to substantially immobilize the first and second syringe clamps after the translating.

When unloading the syringe 206 from the syringe mounting system 200, the user may translate the first and second syringe clamps 210, 212 away from each other such that they no longer contact the rearward portion of the syringe 206 disposed therebetween. In other words, the clamps 210, 212 may be moved from the closed condition to the open condition. This may be accomplished by moving the lever 220 from the closed position shown in FIG. 5 to the open position shown in FIG. 4. Thereafter, the user may move the syringe 206 out from between the first and second syringe clamps 210, 212. Prior to this transition from the closed condition to the open condition, the user may be required to unlock a clamp lock of the syringe mounting system 200 to allow translational movement of the clamps 210, 212 to take place.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A front-loading, contrast media injector comprising:
an injector housing having an opening defined therein;
a drive ram, at least a portion of which is movable into and out of the injector housing along a longitudinal reference axis of the drive ram through the opening in the injector housing; and
a syringe mount coupled to the housing and comprising:
a first syringe clamp comprising a first arcuate surface;
a second syringe clamp comprising a second arcuate surface; and
a lever configured to actuate each of the first and second syringe clamps between a syringe-engaging, closed position and a syringe-disengaging, open position, wherein the lever is movable relative to each of the first and second syringe clamps between first and second lever positions,
wherein the first and second syringe clamps are in opposing relation to one another such that the first arcuate surface faces the second arcuate surface, wherein the first and second syringe clamps translate in opposite linear directions toward and away from one another to engage and disengage a syringe located therebetween, and wherein moving the lever into the first lever position linearly moves each of the first and second syringe clamps to the syringe-disengaging, open position, and wherein moving the lever into the second lever position linearly moves each of the first and second syringe clamps to the syringe-engaging, closed position.

2. The injector of claim 1, wherein an aperture is defined between the first and second syringe clamps to align a plunger of a syringe with the drive ram.

3. The injector of claim 1, wherein each of the first and second syringe clamps comprises a C-clamp.

4. The injector of claim 1, wherein the first and second syringe clamps are configured to open and close about a circumferential portion of a syringe.

5. The injector of claim 4, wherein the first and second syringe clamps are in contact with one another when the same are closed about a circumferential portion of a syringe.

6. The injector of claim 1, wherein the first and second syringe clamps are linearly moved, by actuation of the lever, along a line that is perpendicular to the longitudinal reference axis along which the drive ram is movable.

7. The injector of claim 1, wherein the syringe mount comprises rails, and wherein the first and second syringe clamps move along the rails between the syringe-engaging, closed position and the syringe-disengaging, open position.

8. The injector of claim 1, further comprising an elongate syringe support extending out away from the injector housing.

9. The injector of claim 1, wherein the syringe mount is a component of a removable face plate of the injector.

10. The injector of claim 1, wherein the syringe mount is substantially integral with the injector housing.

11. The injector of claim 1, wherein the syringe mount is a component of an adapter for at least temporarily making an original syringe mount of the injector compatible to accommodate a syringe that the original syringe mount was not originally designed to accommodate.

12. The injector of claim 1, further comprising a syringe disposed between the first and second syringe clamps.

13. The injector of claim 12, further comprising contrast media, saline, or a combination thereof disposed within the syringe.

14. A method of using a powered contrast media injector, the method comprising:
executing a first placing step comprising placing a syringe mounting system in an open condition, wherein the syringe mounting system comprises first and second syringe clamps, and wherein the first placing step comprises linearly moving the first and second syringe clamps away from each other;
disposing a rearward portion of a syringe between the first and second syringe clamps, wherein each of the first and second syringe clamps comprises an arcuate surface, and wherein the disposing step is executed after the first placing step; and
executing a second placing step comprising placing the syringe mounting system in a closed condition, wherein the second placing step is executed after the disposing step and without moving the syringe relative to the injector, and wherein said second placing step comprises linearly moving the first and second syringe clamps toward one another at least until each of the arcuate surfaces contacts the rearward portion of the syringe.

15. The method of claim 14, further comprising:
actuating a lock to substantially immobilize the first and second syringe clamps after execution of the second placing step.

16. The method of claim 14, further comprising:
causing the injector to move a plunger of the syringe while the rearward end of the syringe is disposed between the first and second syringe clamps.

17. The method of claim 14, wherein the second placing step occurs until the first and second syringe clamps are in direct contact with one another.

18. A method of using a powered contrast media injector, the method comprising:
disposing a rearward portion of a syringe between first and second syringe clamps of the injector;
executing a first translating step comprising translating each of the first and second syringe clamps into a syringe-engaging, closed position, wherein the first translating step consists essentially of moving a lever into a first position and relative to each of the first and second syringe clamps;
executing a second translating step comprising translating each of the first and second syringe clamps into a syringe-disengaging, open position, wherein the second translating step consists essentially of moving the lever into a second position and relative to each of the first and second syringe clamps; and
moving the syringe out from between the first and second syringe clamps after the second translating step.

19. The method of claim 18, further comprising:
unlocking a clamp lock to allow for translation of the first and second syringe clamps, wherein the unlocking occurs prior to the second translating step.

20. The method of claim 18, wherein the first and second syringe clamps are in direct contact with one another prior to the second translating step.

21. The method of claim 18, wherein each of the first and second translating steps occurs in a substantially lateral orientation.

22. The method of claim 18, further comprising expelling at least one of contrast media and saline from the syringe.

23. The method of claim 14, wherein each of the first and second placing steps comprises moving a single lever relative to each of the first and second syringe clamps.

24. The method of claim 14, further comprising expelling at least one of contrast media and saline from the syringe.
